(19) United States
(12) Patent Application Publication
(10) Pub. No.: US 2025/0349428 A1
(43) Pub. Date: Nov. 13, 2025

(54) LLM SKILL LEARNING FOR MEDICAL DECISION MAKING THROUGH SELF-PLAY

(71) Applicant: NEC Laboratories America, Inc., Princeton, NJ (US)

(72) Inventors: Wei Cheng, Princeton Junction, NJ (US); Haifeng Chen, West Windsor, NJ (US); Jonathan Li, Troy, NY (US)

(21) Appl. No.: 19/205,380

(22) Filed: May 12, 2025

Related U.S. Application Data
(60) Provisional application No. 63/646,171, filed on May 13, 2024.

(51) Int. Cl. G16H 50/20; G16H 50/70

(52) U.S. Cl. CPC G16H 50/20; G16H 50/70

(57) ABSTRACT

Methods and systems for medical decision making include selecting a strategy from a strategy library, expressed in natural language, and selecting an improvement from an improvement library, expressed in natural language. The strategy is combined with the improvement using a large language model (LLM) to generate an improved strategy. The improved strategy is evaluated to generate feedback. The strategy library and the improvement library are updated based on the feedback. An action is performed based on the improved strategy.

### RELATED APPLICATION INFORMATION

This application claims priority to U.S. Patent Application No. 63/646,171, filed on May 13, 2024, incorporated herein by reference in its entirety.

### BACKGROUND

### Technical Field

The present invention relates to large language model tuning and, more particularly, to tuning an LLM for skills using self-play.

### Description of the Related Art

Pre-trained large language models (LLMs) can have difficulty adapting to specialized tasks without fine tuning using a substantial and relevant dataset. Furthermore, such fine tuning can disrupt the reasoning and planning capabilities of the LLM, which can harm the LLM's performance in applications where decision making in novel situations is needed. These challenges are magnified in multi-agent settings, where complex inter-agent dynamics necessitate advanced communication, deduction, and collaboration.

### SUMMARY

A method for medical decision making includes selecting a strategy from a strategy library, expressed in natural language, and selecting an improvement from an improvement library, expressed in natural language. The strategy is combined with the improvement using a large language model (LLM) to generate an improved strategy. The improved strategy is evaluated to generate feedback. The strategy library and the improvement library are updated based on the feedback. An action is performed based on the improved strategy.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of a skill coach used to provide improvements to strategies, in accordance with an embodiment of the present invention;

FIG. 2 is a block diagram of dialogue generation in the context of a multi-agent environment, in accordance with an embodiment of the present invention;

FIG. 3 is a block/flow diagram of a method for generating dialogue actions and updating a skill coach based on feedback, in accordance with an embodiment of the present invention;

FIG. 4 is a block diagram of a healthcare facility where treatment strategies can be automatically learned and used for medical decision making, in accordance with an embodiment of the present invention;

FIG. 5 is a block diagram of a computing device that can learn treatment strategies, in accordance with an embodiment of the present invention;

FIG. 6 is a diagram of an exemplary neural network architecture that can be used to implement part of a large language model (LLM), in accordance with an embodiment of the present invention;

FIG. 7 is a diagram of an exemplary deep neural network architecture that can be used to implement part of an LLM, in accordance with an embodiment of the present invention.

### DETAILED DESCRIPTION OF PREFERRED EMBODIMENTS

A large language model (LLM) can be used for skill acquisition in a non-parametric approach that provides ongoing improvements to the LLM with minimal human annotation. This can be accomplished using self-play, coupled with a Monte Carlo tree search (MCTS) to simulate scenarios, providing the LLM with quality feedback and generating new data for learning advanced skills such as state evaluation and dialogue generation. LLM-powered agents can then outperform reinforcement learning approaches. This provides better training in complex interactive environments, illustrating the potential for LLM agents in customer support, medical decision making, and other applications that make use of sophisticated communication strategies.

To that end, a skill coach is implemented that uses LLMs to learn new skills. The skills herein refer to any high-level strategy or tactic that can be learned. For example, in a game setting, a value heuristic may be learned to evaluate different states of the game and a textual strategy guide may be generated on how to create dialogue. High-level skills are targeted to provide high-level strategic planning, abstracting away low-level details that may be scenario specific. While the skill coach helps to learn high-level strategies, an evaluator executes and evaluates the high-level strategies on the lower level. This can be accomplished using simulations and self-play, where the evaluator compares agents who use different strategies against one another.

The skill coach maintains a strategy library of the strategies it has generated so far, starting from seed strategies, along with performance scores and raw feedback of how the strategies performed in practice. The skill coach also maintains a library of improvement ideas, which can be understood as ways to improve the strategies in different circumstances, along with how much the idea improves the strategies' performance on average.

Referring now to FIG. 1, a block diagram illustrating a skill coach 100 is shown. The skill coach 100 includes idea generation 110, where a sampled strategy and feedback are fed into a feedback interpreter 112 to select what parts of the feedback to include. The feedback interpreter 112 converts these inputs to a natural language form. In some embodiments the feedback interpreter 112 may use an LLM to analyze the input feedback.

The natural language output of the feedback interpreter 112 is used as input to idea generator 114, which generates a new improvement using an LLM. The improvement is stored in an improvement library 130. For example, the idea generator 114 may prompt the LLM to ask for ways to improve a given strategy based on the feedback from the feedback interpreter 112.

Strategy improvement 140 samples a strategy from strategy library 120, along with one of the improvements in the improvement library 130. The improvement library 130 may be sampled using a bandit algorithm. This strategy/improvement may be selected according to a performance score associated with each in their respective libraries. The selected strategy and improvement are input to an implementer 142, which improves the strategy using the improvement, generating an improved strategy. For example, implementer 142 may prompt the LLM to implement the improvement into the strategy, expressed in natural language.

Evaluator 144 then evaluates the improved strategy output by the implementer 142. This may include testing the improved strategy on low-level test cases and scenarios, recording feedback on how the improved strategy performed as well as an average performance score for the strategy. As used herein, the term “low-level” refers to a detailed step, while “high-level” refers to the general strategy. The improvement library 130 is updated with a value indicating how much the idea improved the strategy and the improved strategy, along with its feedback and score, are added to the strategy library 120.

It can be difficult to evaluate whether an improved strategy is better than the original strategy and why. Simply prompting the LLM to improve the strategy based on the feedback, without guidance from the improvement, can result in the LLM making changes to all parts of the strategy. After evaluating such a strategy, it can be difficult to determine which specific change resulted in the corresponding change in performance. The improvement library 130 helps by focusing the LLM's changes to a particular area, modularizing the search process. Instead of improving every part of the strategy at once, the improvements make incremental changes, which can be used to identify which particular changes resulted in a benefit without confounding factors.

The improvement library 130 further helps to track successful improvements, which may be helpful in other circumstances in the future. Sometimes a given improvement can be applied multiple times to a given strategy to achieve better performance. Other times, an idea generated from feedback from one strategy can also be helpful when applied to another strategy. Upper confidence bound sampling from the improvement library 130 helps to both explore new improvements and to exploit old ones.

Another challenge lies in how to generate quality feedback and how to convert that feedback into natural language that the LLM can process. Without good feedback, the LLM lacks guidance on what improvements might work and will blindly test out ideas instead.

A goal is to learn a good policy function, which can be accomplished by improving strategies associated with the policy function. Given some state space S and some action space [symbol] , a policy function ϕ in a policy space Φ is a mapping ϕ:S→Δ [symbol] , where ϕ may output a probability distribution over the actions Δ [symbol] . An environment ε=<S, [symbol] , [symbol] , N, T, R, A, ϕₑ> defines the state space S, the action space [symbol] , a set of agents [symbol] , a transition function T:S, [symbol] →S, a reward function R:S, [symbol] → [symbol] that specifies intermediate rewards for each agent, an action function A:S→ [symbol] , [symbol] ( [symbol] ) that defines which agent may take what legal actions at some state, where [symbol] is a power set, and ϕₑ as a policy function for an environment agent. The transitions are deterministic, and stochastic transitions are handled by the environment agent e∈ [symbol] . For a partial information environment, a function H:S, [symbol] → [symbol] maps from hidden states and actors to hidden information sets [symbol] . Hence ϕ: [symbol] →Δ [symbol] is a function from information sets to action distributions. A strategic profileϕ=[ϕᵢ]_{|} [symbol] _{|} describes the policies for every player i∈ [symbol] .

A function ƒ:Σ→Φ maps strategies to policies. A high-level strategy σ∈Σ helps parameterize policies to search over the lower-dimensional Σ space instead of Φ. Letting Φ₋ᵢ denote the space of possible opponent policies, where −i are the indices of players other than i, the goal becomes finding an optimal strategy σᵢ that approximates the optimal policy given the policies of the other agents ϕ₋ᵢ:

$${argmax}_{{σ}_{i}} {τ ∼ ( f ( {σ}_{i} ) , {σ}_{- i} )}_{} [ {\sum }_{( s , a ) ∈ τ} {R}_{i} ( s , a ) ] \approx {argmax}_{τ ∼ ( {ϕ}_{i} , {ϕ}_{- i} ) )} [ {\sum }_{( s , a ) ∈ τ} {R}_{i} ( s , a ) ]$$

where τ=(s₀, α₀, . . . ) is a simulated trajectory according to the strategic profile (ϕᵢ,ϕ₋ᵢ) and the transition function T, with αₜ˜ϕ(αₜ|sₜ) and sₜ₊₁=T(sₜ,αₜ). Thus the probable strategic profile of other players may be learned, for example by assuming that they play optimally.

The state space and action space are different depending on the setting. For example, in a card game, the state space S may simply include the cards played so far and the action space [symbol] might include the cards that can be played. In a medical context, the state space S may include a patient's medical history and recent biometric information such as heart rate, blood pressure, and blood oxygen saturation. In such a context, the action space [symbol] may include the possible treatments that can be performed.

The action space may furthermore be separated into categories. Following the medical example, the actions may be divided into exemplary categories of tests and treatments, where tests obtain more information and treatments perform some action to change the patient's health state. Each category may have its own analysis and action generation components, to analyze the current state of the patient and to generate plans. The testing actions may be used to update the state of the patient.

Thus both high-level strategies and low-level skills may be learned to treat a patient automatically, such as by determining what medicine to give the patient and at what dosage. These strategies can be learned from the feedback of doctors and can be summarized into a natural language output or into value heuristic code for different diseases and conditions.

Features of the policies ϕ may be abstracted as high-level strategies σ, which are more suitable to handling by an LLM. A strategy σ may be executed and refined by a low-level executor, for example during inference, resulting in an execution policy ϕ_{σ}:
feedback→LLM Improver→σ→Executor(σ)→ϕ_{σ}
   Abstraction offers benefits in that high-level strategies provide a more abstract, compressed, and intuitive representation of the problem, which an LLM can process, thereby providing generalization and reasoning capabilities. Furthermore, searching over high-level strategies is more efficient than low-level policy exploration, as it simplifies the search space by focusing on core principles, rather than fine details. This guides the search toward promising areas more quickly, leveraging domain knowledge, patterns, and/or heuristics that generalize across scenarios.

At the low level, the strategy can be executed by selecting the action that leads to the best state:

$${ϕ}_{i} ( s ) = Q ( s , a ) = {R}_{i} ( s , a ) + {v}_{i} ( {s}^{′} ) \vert {s}^{′} = T ( s , a ) .$$

Since the value νᵢ is learned for each agent, the best action for each agent I can be determined, refining the value function into the strategic profile ϕ. Value heuristics can be inaccurate, so the policy search may be refined using MCTS to look ahead through multiple action-state sequences to provide a better policy. MCTS also generates additional feedback by comparing the updated value estimated from MCTS with the initial value heuristic. The estimated win rate from the search provides a shaped reward signal, which is more informative than the simple win/lose outcome reward.

Idea generator 114 selects a strategy σ and its feedback trajectory τ_{σ} using an adaptive selection policy. The feedback may include trajectories from previous self-play simulations, including visited states, actions taken, estimated win rates, final outcomes, and intermediate values. To avoid processing lengthy trajectories, key states may be selected that best capture discrepancies between the strategy's value heuristic and search-based estimates. These key states are translated into natural language and used to prompt the LLM for new improvement ideas. The new improvements are added to a queue with a prior score estimating their potential effectiveness.

When performing implementation 142, a strategy σ is samples from the strategy library 120 and an improvement d is sampled from the improvement library 130, for example using a sampling method that balances exploration and exploitation. The LLM refines σ using d to generate a new strategy σ_{new}. The new strategy is implemented and evaluated using self-play simulations, which produces win rates W[σ] and trajectory feedback [symbol] [σ]. During simulations, agents conduct MCTS tree searches and estimate win rates at different states, providing additional feedback. The strategy library 120 is updated with the new strategy and its performance. The improvement score of the idea d is updated based on how much it improved the performance of σ.

The improvement library 130 helps to refine strategies incrementally, rather than globally, to avoid confounding factors and ensure interpretability. The improvement library 130 may be implemented as a queue, and upper confidence bound (UCB) sampling may be used to balance exploration of new improvements and exploitation of proven ones:

$$UCB ( idea ) = {\overline{z}}_{idea} + c \sqrt{ln ( {N}_{total} ) / {N}_{idea}}$$

where z̅ is the empirical average improvement score, Nₜₒₜₐₗ is the total number of improvements implemented, and N_{idea} is the number of implementations of the specific improvement. The queue tracks successful improvements that are generalizable across strategies, enabling transfer and reuse of improvements. Improvements are often additive, providing penalties or adjustments, and enhance performance when applied to similar strategies.

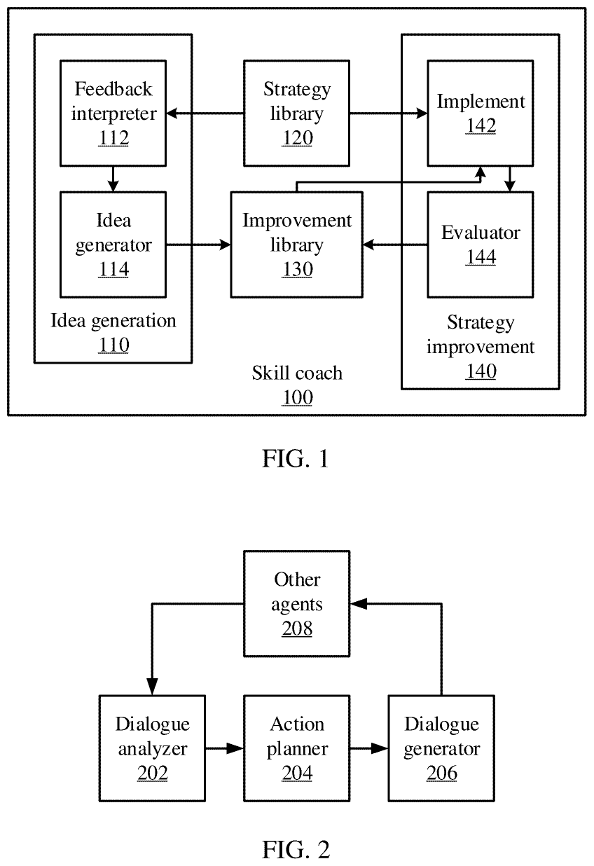

Referring now to FIG. 2, a diagram shows the interaction of different categories of action. In some applications, a dialogue-based environment may include the ability to interact with other agents and move within the environment. The categories may thus include dialogue generation and movement, with the two parts being integrated to produce an agent that is capable of interacting with the environment using dialogue.

A language component may include a dialogue analyzer 202 and a dialogue generator 206, while the movement may be controlled by an action planner 204. Whenever the agent needs to speak, they first analyze what was said so far in the current conversation using the dialogue analyzer. The dialogue analyzer 202, with the help of an LLM, updates the internal beliefs of the agent. For example, internal beliefs may include a probability that the agent assigns to each other agent regarding their expected intentions and status.

These beliefs are then passed to the action planner 204, which uses them to determine an action intent. The action intent is used by the dialogue generator 206 to generate dialogue using the LLM. When the agent needs to move, the same process is used, except the agent performs the action intent and no dialogue is generated. After interactions with the other agents 208, their dialogue responses are fed back into the dialogue analyzer to determine the next step.

For non-dialogue actions, while the action spaces and state spaces themselves are usually discrete and finite, the number of possible functions from state space to action space is very large. Whereas reinforcement learning might tackle the problem of a large policy space by parameterizing the model and optimizing the parameters instead, the use of an LLM for skill learning can help to search and optimize over the policy space more effectively. Given rules of the environment in natural language form, the LLM can quickly generate reasonable policies. It is often easier to describe the value of a state in natural language form, versus describing the optimal action to take in a given state, since the optimal action is often described by comparing the options. A value function is learned instead. It is easy to convert from a value function to a policy function by taking the action that leads to the best state. Instead of learning the policy function directly, the LLM can be used to learn a value heuristics function instead. The value heuristic function may be generated program code which takes state information as input (e.g., strings) and outputs a value. A better policy will help to generate a better function, which creates better estimates.

The value heuristics may be expressed as σ:=ν:S→ [symbol] to parametrize the policy ϕ, estimating an expected cumulative return for each player at a given state. Using a value heuristic simplifies reasoning, as it is easier to describe how good a state is than it is to specify an optimal action. This makes it intuitive for the LLM to reason about winning probabilities.

The value heuristics function may provide an inaccurate estimate of the unknown true value function. To resolve these inaccuracies, the policy function may be enhanced with MCTS. A trajectory is simulated from a current hidden state to some unexpanded state s. The probability of transitioning to a state during simulations is determined assuming that each agent samples from their optimal actions according to their polynomial upper confidence tree (PUCT) values, including ϕₑ for the environment agent.

Because some agents may only be able to observe information sets, the PUCT values may be averaged over all expanded states in the information set. The initial hidden state can be sampled according to a prior, or empirical prior, over the states in the information set that the agent has observed. Using the value heuristic, the values of each of the next hidden states may be calculated and backpropagated back up the simulated trajectory, updating the intermediate states. After running some MCTS simulations, the action planner 204 outputs the action which leads to the highest-value next state.

This search process provides better value estimates than those which are initially given by the value heuristic when making decisions. In addition, the search process makes it possible to generate more feedback than would otherwise be possible, as the updated value estimate computed through MCTS can be compared with the initial estimate from the value heuristic.

The PUCT can be expressed as:

$$Q ( s , a ) = \frac{N ( s , a ) \cdot {Q}_{emp} ( s , a ) + α \cdot \hat{Q} ( s , a )}{N ( s , a ) + α}$$

$$PUCT ( I , a ) = {\sum }_{s ∈ I} {π}_{B} ( s \vert I ) [ Q ( s , a ) + C \cdot P ( S , a ) \cdot \frac{\sqrt{{\sum }_{b} N ( s , b )}}{1 + N ( s , a )} ]$$

where P(s,α) is a prior probability of selecting action a from state s, N(s,α) is a number of times the action a was selected at state s during MCTS rollouts, C is an exploration constant, Qₑₘₚ is the empirical average of MCTS rollout outcomes, \hat{Q}(s,α) is a prior computed by the value heuristic, α controls how much weight is put on the prior, and π_{B} is a distribution across hidden states in an information set I given a set of beliefs B, some parameterization of π_{B}. Since π_{B} can be difficult to compute, it can be set to:

$${π}_{B} ( s \vert I ) = \frac{{\sum }_{b} N ( s , a )}{{\sum }_{{a}^{′} ∈ I} {\sum }_{b} N ( {s}^{′} , b )}$$

to be the empirical rollout distribution, given that initial states are sampled s₀˜π_{B}(sₒ|I) according to the beliefs. The information set I is a set of text descriptions of current status and conditions (states) and strategies. The value b is a sampled belief.

The LLM can be prompted for actions or values given the state. However, this method may be costly, as the LLM needs to generate both thoughts and moves. When the search is used, the value heuristic may be queried multiple times to make a single move, and it may not be feasible to query the LLM that many times. Instead, the LLM can be used to analyze and improve upon formally written value heuristic functions. Specifically, the LLM can be prompted to write the value heuristic in the form of programming code so that it is easier to verify and execute, and also easier for the LLM to reason and improve on it given the formal structure of the code.

In dialogue generation, both the action space and the state space can be very large. For example, the action space may include the number of possible sequences of words that could be generated for a discussion round, and the state space may include the number of possible responses from the previous round. This means that the number of possible dialogue generation policies is very large, and that parameter optimization approaches will have difficulty optimizing across the space.

The present embodiments instead learn a high-level strategy guide for the dialogue generator 206. The strategy guide formalizes a process for dialogue generation in a given situation. This may be implemented in question-and-answer form, where the strategy guide contains the questions. The LLM in the dialogue generator may be prompted to answer all the questions in the strategy guide before using it as a prompt to generate dialogue.

It can be difficult to provide good reward signals during the dialogue training process. One problem is a lack of training data. Existing dialogue generation methods often rely on imitation learning on existing human generated text data through parameter training of the underlying network. However, in many settings, such large quantities of human generated data might not be readily available for the tuning of language models. The present embodiments only need one game's worth of simulated dialogue.

Given the scenarios from the training data, a second problem lies in how to accurately evaluate how well generated dialogue performs. Agents need to optimize and balance multiple objectives when discussing, and there may be no clear metric. For example, an agent may have multiple goals that they are working toward simultaneously. The most accurate way to acquire the true reward signals is to simulate many interactions similar to how signals were acquired for the action planner before and take the average performance as the evaluation metric. However, simulating dialogue can be costly, since the LLM may need to be prompted for all agents multiple times for each other agent.

To address this problem, a scenario is simulated using some initial policy π₀ and the simulation is stored in a scenario database. Then during evaluation, one scenario is pulled from the database to evaluate with. A scenario is simply a decision point when an agent had to generate dialogue. The dialogue generator 206 is given a history of the previous discussions and moves up to the decision point as if it conducted the dialogue up to that point. The dialogue generator 206 is prompted to generate new dialogue using the new strategy guide. The beliefs of the other players are updated using the dialogue analyzer 202, and proceed to continue the simulation assuming no other dialogue happens, using the action planner 204 only. The existing scenario database can be bootstrapped by adding in the new dialogue generated during the improvement process.

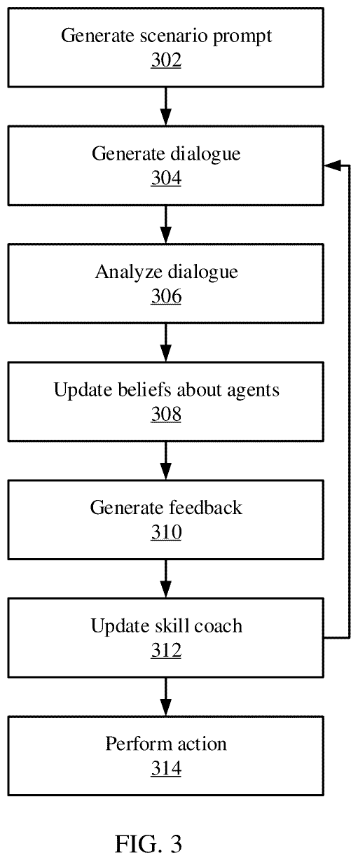

Referring now to FIG. 3, a method for dialogue generation is shown. Block 302 begins by generating a scenario prompt, for example providing the LLM with the current state and a summary of previous dialogue. The scenario prompt can further state information about the agent's role and information that it has access to, along with information about the agent's goals and an intended action.

Block 304 then uses the dialogue generator 206 to generate dialogue, given the current scenario, to achieve the agent's goals. The generation of the dialogue may make use of strategies from skill coach 100. Such strategies may, for example, include recommendations on how to present information in the dialogue to achieve an intended goal. Block 304 may deliver the generated dialogue using the agent.

Block 306 analyzes any responses to the generated dialogue using dialogue analyzer 202, for example with a prompt that asks whether the response affected the agent's goals. The response may provide information that increases the agent's certainty regarding a particular goal in the context of the situation. Block 308 updates the agent's beliefs about the other agents, for example updating likelihoods that the other agents possess certain knowledge or traits.

This can be used to generate feedback in block 310, for example in a natural language report that describes the outcome of the dialogue. This feedback information can then be used in block 312 to update the skill coach 100 as described above. This process may be repeated to build out the strategy library 120 and the improvement library 130. At inference time, block 314 may be used to perform an action for a given state, using the previously identified strategies and improvements.

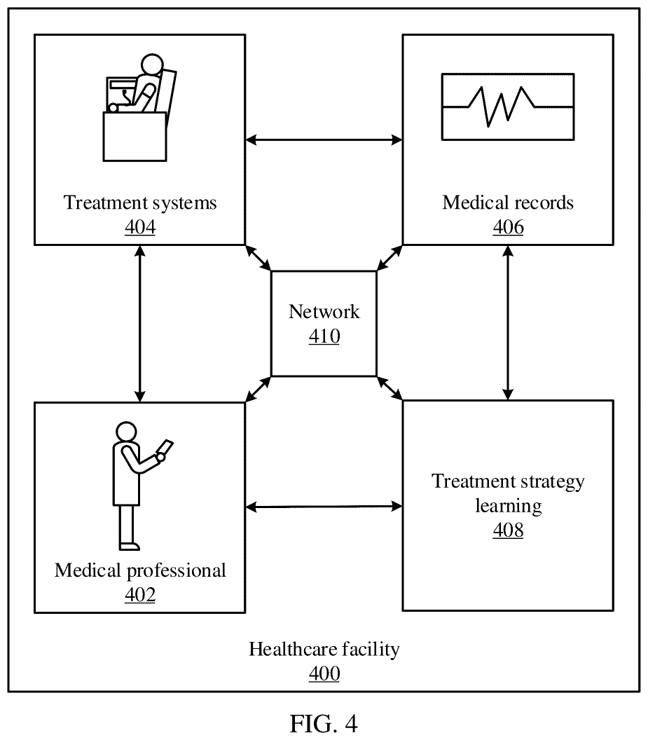

Referring now to FIG. 4, a diagram of information extraction is shown in the context of a healthcare facility 400. Treatment strategy learning 408 may be used to generate a custom treatment for a patient, responsive to information gleaned from tests and the patient's medical records 406. This information represents the state of the patient, and treatment strategy learning 408 can be used to generate a policy that determines a treatment action based on the state.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Medical professionals 402 may use treatment strategy learning 408 to provide customized healthcare that is tailored to the patient's needs. For example, the medical professionals 402 may use treatment strategy learning 408 to identify an action that will provide the best improvement to the patient's health state.

The different elements of the healthcare facility 400 may communicate with one another via a network 410, for example using any appropriate wired or wireless communications protocol and medium. Thus the treatment strategy learning 408 can be used to design a treatment that targets a patient's specific condition, for example using test results and medical records 406. The treatment systems 404 may be used to generate and administer a therapy based on treatment strategy learning 408.

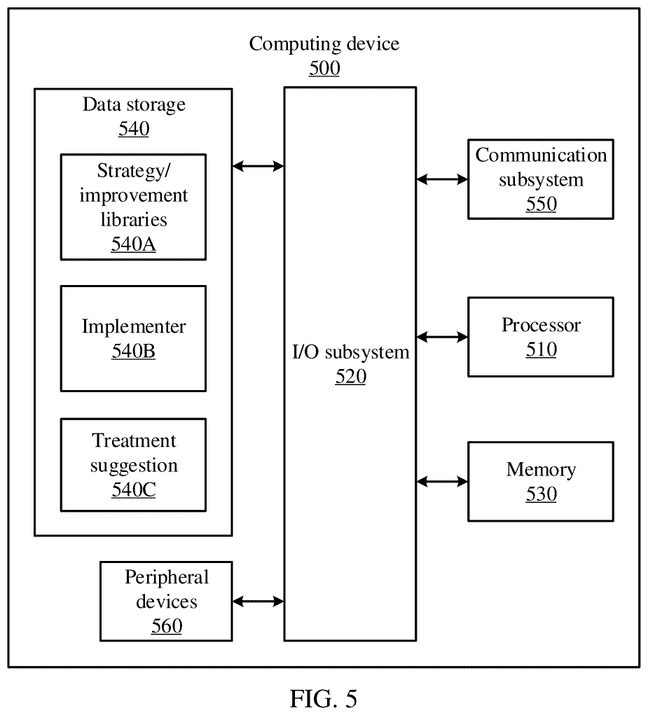

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for strategy and improvement libraries, 540B for implementing strategies and improvements to evaluate new combinations, and/or 540C for suggesting treatments based on learned strategies. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

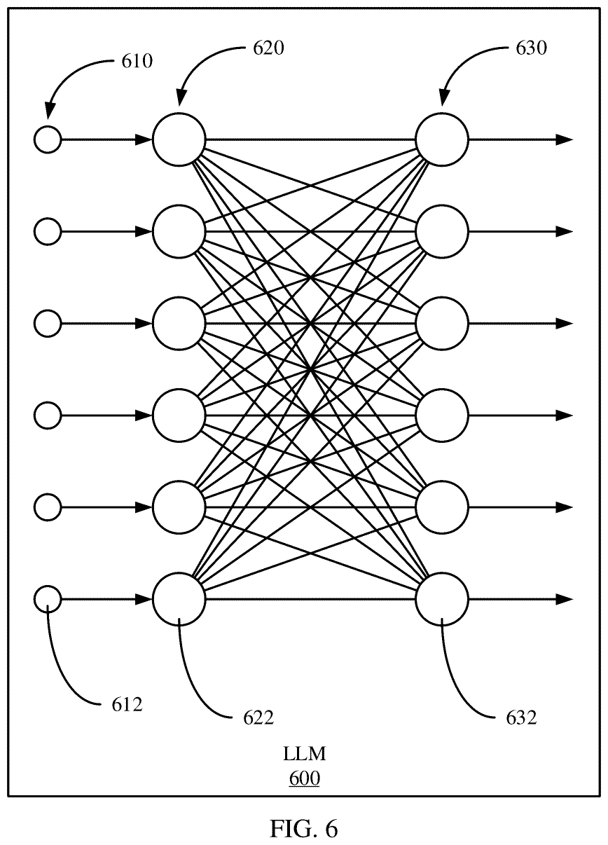

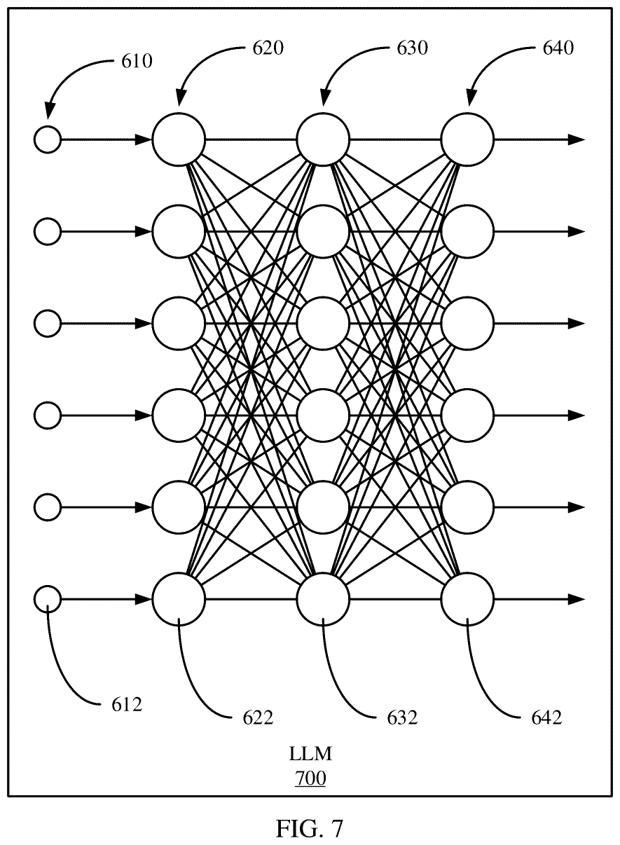

Referring now to FIGS. 6 and 7, exemplary neural network architectures are shown, which may be used to implement parts of the present models, such as the LLM 600/700. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for medical decision making, comprising:
selecting a strategy from a strategy library, expressed in natural language;
selecting an improvement from an improvement library, expressed in natural language;
combining the strategy with the improvement using a large language model (LLM) to generate an improved strategy;
evaluating the improved strategy to generate feedback;
updating the strategy library and the improvement library based on the feedback; and
performing an action based on the improved strategy.

2. The method of claim 1, further comprising adding a new improvement to the improvement library by prompting the LLM to suggest an improvement for the strategy.

3. The method of claim 1, wherein the action includes generating dialogue using the LLM in accordance with the improved strategy.

4. The method of claim 3, wherein evaluating the improved strategy includes updating information about a state of another agent in a scenario.

5. The method of claim 3, wherein evaluating the improved strategy includes generating a scenario prompt that includes agent goals.

6. The method of claim 1, wherein the improvement library includes a set of improvements, each associated with a score that reflects how it affects performance.

7. The method of claim 1, wherein the strategy relates to treating a medical condition and wherein the action includes automatically performing a treatment action on a patient.

8. The method of claim 1, wherein the LLM is implemented using a machine learning model.

9. The method of claim 1, wherein evaluating the improved strategy includes performing a Monte Carlo tree search over a strategy tree.

10. The method of claim 1, wherein selecting the improvement includes selecting a plurality of improvements, and wherein combining the strategy includes combining the strategy with all of the plurality of improvements.

11. A system for medical decision making, comprising:
a hardware processor; and
a memory that stores a computer program which, when executed by the hardware processor, causes the hardware processor to:
select a strategy from a strategy library, expressed in natural language;
select an improvement from an improvement library, expressed in natural language;
combine the strategy with the improvement using a large language model (LLM) to generate an improved strategy;
evaluate the improved strategy to generate feedback;
update the strategy library and the improvement library based on the feedback; and
perform an action based on the improved strategy.

12. The system of claim 11, wherein the computer program further causes the hardware processor to add a new improvement to the improvement library by prompting the LLM to suggest an improvement for the strategy.

13. The system of claim 11, wherein the action includes generating dialogue using the LLM in accordance with the improved strategy.

14. The system of claim 13, wherein the computer program further causes the hardware processor to update information about a state of another agent in a scenario.

15. The system of claim 13, wherein the computer program further causes the hardware processor to add generate a scenario prompt that includes agent goals.

16. The system of claim 11, wherein the improvement library includes a set of improvements, each associated with a score that reflects how it affects performance.

17. The system of claim 11, wherein the strategy relates to treating a medical condition and wherein the action includes automatically performing a treatment action on a patient.

18. The system of claim 11, wherein the LLM is implemented using a machine learning model.

19. The system of claim 11, wherein the computer program further causes the hardware processor to perform a Monte Carlo tree search over a strategy tree.

20. The system of claim 11, wherein selection of the improvement includes selection of a plurality of improvements, and wherein combination of the strategy includes combination of the strategy with all of the plurality of improvements.
